(19) United States
(12) Patent Application Publication
(10) Pub. No.: US 2007/0142346 A1
(43) Pub. Date: Jun. 21, 2007

(54) Method of treating breast cancer using 17-AAG or 17-AG or a prodrug of either in combination with a HER2 inhibitor

(76) Inventors: Robert G. Johnson JR., Lafayette, CA (US); Alison L. Hannah, Sebastopol, CA (US); Gillian F. Cropp, San Francisco, CA (US); Yiqing Zhou, Orange Village, OH (US); J. Michael Sherrill, Danville, CA (US)

Correspondence Address: KOSAN BIOSCIENCES, INC, 3832 BAY CENTER PLACE, HAYWARD, CA, 94588, US

(21) Appl. No.: 11/542,960

(22) Filed: Oct. 3, 2006

Related U.S. Application Data
(60) Provisional application No. 60/731,143, filed on Oct. 28, 2005.
(60) Provisional application No. 60/748,987, filed on Dec. 7, 2005.

(51) Int. Cl. A61K 31/395

(52) U.S. Cl. 514/183

(57) ABSTRACT

A method for treating a breast cancer in a subject by administering 17-allylamino-17-demethoxy-geldanamycin (17-AAG) or 17-amino-17-demethoxygeldanamycin (17-AG), or a prodrug of either 17-AAG or 17-AG, in combination with a HER2 inhibitor.

### CROSS-REFERENCE TO RELATED APPLICATIONS

This application claims the benefit under 35 U.S.C. §119(e) of U.S. Provisional Applications Nos. 60/731,143, filed Oct. 28, 2005, and 60/748,987, filed Dec. 7, 2005, the disclosures of which are incorporated herein by reference.

### BACKGROUND OF THE INVENTION

1. Field of the Invention

This invention relates to a method of treating breast cancer using 17-allylamino-17-demethoxygeldanamycin (17-AAG) or 17-amino-17-demethoxygeldanamycin (17-AG), or a prodrug of either, in combination with a HER2 inhibitor.

2. Description of Related Art

Breast cancer, a malignant tumor arising from mammary cells, is the second leading cause of cancer deaths in women worldwide. Most breast cancers begin in the cells that line the ducts or lobules of the breast. The cancer can spread (metastasize) to other organs via the veins and lymphatic vessels, typically the axillary lymph nodes, internal mammary nodes, and/or supra- or infraclavicular nodes.

Therapies for breast cancer include surgery, radiation therapy, and chemotherapy. Chemotherapy agents include doxorubicin (Adriamycin®), cyclophosphamide (Cytoxan®), epirubicin (Ellence(®), gemcitabine (Gemzar®), vinorelbine (Navelbine®), paclitaxel (Taxol®), docetaxel (Taxotere®), capecitabine (Xeloda®), platinum complex drugs (e.g., cisplatin and carboplatin), etoposide, vinblastine, fluorouracil, and trastuzumab (Herceptin®). Combination treatments can be used: cyclophosphamide/methotrexate/fluorouracil (CMF), fluorouracil/doxorubicin/cyclophosphamide (CAF/FAC), doxorubicin/cyclophosphamide (AC), cyclophosphamide/epirubicin/fluorouracil (CEF), epirubicin/cyclophosphamide (EC), docetaxel/doxorubicin/cyclophosphamide (TAC), doxorubicin followed by CMF (A→CMF), and AC followed by paclitaxel (AC→T). Chemotherapy can include hormonal treatment with an estrogen blockers such as tamoxifen and fulvestrant.

Most if not all of these drugs have serious side effects or other limitations. Their use may cause loss of appetite, nausea, vomiting, mouth sores, hair loss, and changes in the menstrual cycle. These drugs may also cause a decline in white blood cells (increasing the risk of infection), platelets (causing difficulties related to blood clotting), and red blood cells (leading to fatigue and anemia). Doxorubicin and epirubicin may cause heart damage. Trastuzumab patients who experience a remission of the cancer suffer a high risk of relapse once they stop taking trastuzumab. Hormonal treatment carries risks; for example, tamoxifen may increase the risk of endometrial cancer and stroke, and fulvestrant may cause gastrointestinal symptoms, headache, back pain, vasodilation, pharyngitis, and vaginal bleeding.

Because none of the existing therapies for breast cancer offer a significant potential for a lasting cure for the disease for most patients, intensive efforts are underway in many research laboratories and clinical centers throughout the world to find a treatment that is truly efficacious.

A variety of compounds are currently being investigated for use in the treatment of breast cancer. One such compound is 17-allylamino-17-demethoxygeldanamycin (“17-AAG”, non-proprietary name tanespimycin, also sometimes referred to as 17-allylamino-geldanamycin). 17-AAG is a semi-synthetic analog of the natural product geldanamycin (Sasaki et al. 1981), which in turn is obtainable by culturing a producing organism, such as Streptomyces hygroscopicus var. geldanus NRRL 3602. Another biologically active geldanamycin derivative is 17-amino-17-demethoxygeldanamycin (“17-AG”), which is produced in the human body by metabolism of 17-AAG (Egorin et al. 1998). 17-AG can also be made by chemical synthesis from geldanamycin (Sasaki et al. 1979). While geldanamycin and its analogs have been studied intensively as anti-cancer agents in the 1990s (e.g., Sasaki et al. 1981; Schnur 1995, Schnur et al. 1995a and 1995b; Schnur et al. 1999), none of them has been approved for anti-cancer use.

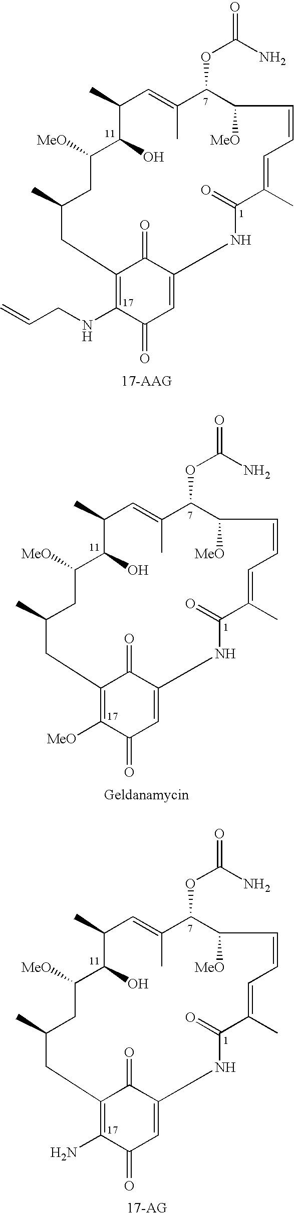

17-AAG and geldanamycin are believed to act by binding to and inhibiting the activity of heat shock protein-90 (“Hsp90”) (Schulte and Neckers, 1998). Hsp90 acts as a chaperone for the normal processing—especially the correct folding—of many cellular proteins (“client proteins”) and is found in all mammalian cells. Stress (hypoxia, heat, etc.) induces a several-fold increase in its expression. There exist other stress-induced proteins (co-chaperones), such as heat shock protein-70 (“Hsp70”), which also play a role in cellular response to and recovery from stress.

In cancer cells, Hsp90 inhibition leads to disruption of the interaction between Hsp90 and its client proteins, such as erbB2, steroid receptors, raf-1, cdk4, and Akt. For example, exposure to 17-AAG results in depletion of erbB2 and destabilization of Raf-1 and mutant p53 in SKBr3 breast cancer cells (Schulte and Neckers, 1998), depletion of steroid receptors in breast cancer cells (Bagatell et al. 2001), depletion of Hsp90 and down-regulation of Raf-1 and erbB2 in MEXF 276L melanoma cells (Burger et al. 2004), depletion of Raf-1, c-Akt, and Erk1/2 in colon adenocarcinoma cells (Hostein et al. 2001), down-regulation of intracellular Bcr-Abl and c-Raf proteins and reduction of Akt kinase activity in leukemia cells (Nimmanapalli et al. 2001), degradation of cdk4, cdk6, and cyclin E in lung cancer cells with wild-type Rb (Jiang and Shapiro 2002), and depletion of erbB1 and erbB2 levels in NSCLC cells (Nguyen et al. 2000).

Johnson, Jr., et al., U.S. patent applications Ser. No. 11/412,298, filed Apr. 26, 2006, and Ser. No. 11/412,299, filed Apr. 26, 2006, disclose the use of Hsp90 inhibitors such as 17-AAG for the treatment of multiple myeloma, as a single agent and in combination with a proteasome inhibitor, respectively.

Because of the activity of 17-AAG relative to Hsp90 and other proteins involved in oncogenesis and metastasis of cancer cells, a number of clinical investigators have evaluated its effectiveness as an anti-cancer agent in human clinical trials. From these various trials, the Cancer Therapy Evaluation Program (CTEP) of the National Cancer Institute recommended these Phase 2 dose/schedule regimens for further study: 220 mg/m² (mg per square meter of body surface area (BSA) of the patient or subject) administered twice weekly for 2 out of 3 weeks, 450 mg/m² administered once a week continuously or with a rest or break, and 300 mg/m² once a week for 3 weeks out of 4 weeks. Results of various clinical trials—almost exclusively with patients having solid tumors—with 17-AAG generally showed limited clinical activity and are summarized below:
(a) A Phase 1 trial in adult patients with solid tumors was conducted in which patients received 17-AAG daily for 5 days every 3 weeks. The starting dose was 10 mg/m² and was escalated to 56 mg/m², with a maximum tolerated dose (“MTD”) and recommended Phase 2 dose defined as 40 mg/m². The protocol was amended to exclude patients with significant pre-existing liver disease, after which patients were treated at doses up to 110 mg/m² on the same schedule. No objective tumor responses were observed. Due to dose limiting reversible hepatotoxicity, the protocol was further amended to dose patients on a twice weekly schedule every other week starting at a dose of 40 mg/m² per day. At daily doses of 40 and 56 mg/m² for 5 days, the peak plasma concentrations were 1,860±660 and 3,170±1,310 nM, respectively. For patients treated at 56 mg/m² average AUC values for 17-AAG and 17-AG were 6,708 and 5,558 nM*h, respectively, and average t_{1/2} 3.8 and 8.6 hours, respectively. Clearances of 17-AAG and 17-AG were 19.9 and 30.8 L/h/m², respectively, and V_{z} values were 93 and 203 L/m², respectively (Grem et al. 2005; Wilson et al. 2001).
(b) In a second Phase 1 trial, patients with advanced solid tumors received 17-AAG on a daily ×5 schedule at a starting dose of 5 mg/m². At the 80 mg/m², dose limiting toxicities (hepatitis, abdominal pain, nausea, dyspnea) were observed but dose escalations nevertheless were continued until the dose reached 157 mg/m²/day. Further dose schedule modifications were implemented to allow twice weekly dosing. At the 80 mg/m² dose level, the t_{1/2} was 1.5 hours and the plasma Cₘₐₓ was 2,700 nM. Similarly, for 17-AG the t_{1/2} was 1.75 hours and the Cₘₐₓ was 607 nM. Plasma concentrations exceeded those needed to achieve cell kill (10-500 nM) in in vitro and in vivo xenograft models (Munster et al. 2001).
(c) A Phase 1 trial of 17-AAG was conducted in which patients with advanced solid tumors were treated weekly for 3 out of every 4 weeks at a starting dose of 10 mg/m², with a recommended Phase 2 dose of 295 mg/m² . Dose escalations reached a dose of 395 mg/m², at which nausea and vomiting secondary to pancreatitis and grade 3 fatigue were observed. The dosing schedule was amended to allow dosing twice weekly for 3 out of every 4 weeks and twice weekly for 2 out of every 3 weeks. A population pharmacokinetic (PK) analysis was performed on data obtained from this trial. The Vd (volume of distribution) for 17-AAG was 24.2 L for the central compartment and 89.6 L for the peripheral compartment. Clearance values were 26.7 L/h and 21.3 L/h for 17-AAG and 17-AG, respectively. Metabolic clearance indicated that 46.4% of 17-AAG was metabolized to 17-AG. No objective tumor responses have been observed in this trial to date. (Chen et al. 2005).
(d) Another Phase 1 trial in patients with solid tumors and lymphomas was conducted using a weekly dosing for 3 weeks out of a 4 week cycle. The starting dose was 15 mg/m² . Dose escalation reached 112 mg/m² without significant toxicity and was continued with an objective of reaching a dose range of “biological” activity. The MTD for weekly 17-AAG was reached at 308 mg/m². No objective tumor responses have been observed to date in this trial, and the levels of Hsp90 client proteins measured were unchanged during therapy. No correlation between chaperone or client protein levels and 17-AAG or 17-AG PK was seen. There was also no correlation between the 17-AAG PK and its clinical toxicity (Goetz et al. 2005).
(e) Another Phase 1 trial was conducted using a once weekly administration schedule, including 11 patients with metastatic melanoma. The starting dose was 10 mg/m², and dose limiting toxicity was observed at 450 mg/m²/week (grade ¾ elevation of AST). At higher doses (16-450 mg/m²/week) the 17-AAG formulation employed contained 10-40 mL dimethylsulfoxide (DMSO) in a single infusion, which likely contributed to the gastrointestinal toxicity that was observed in the trial. Among the patients treated at 320-450 mg/m², two showed radiologically documented long term stable disease. No complete or partial responses were recorded. At the highest dose level (450 mg/m²) the plasma 17-AAG concentrations exceeded 10 μM and remained above 120 nM for periods in excess of 24 hours. At the highest dose level of 450 mg/m², the mean volume of distribution was 142.6 L, mean clearance was 32.2 L/h, and the mean peak plasma level was 8,998 μg/L. There was a linear correlation between dose and area under the curve (AUC) for the dose levels studied. Pharmacodynamic (PD) parameters were also measured and induction of the co-chaperone protein Hsp70 was observed in 8 of 9 patients treated at 320-450mg/m²/week. Depletion of client proteins was also observed in tumor biopsies: CDK4 in 8 out of 9 patients and Raf-1 depletion in 4 out of 6 patients at 24 hours. These data indicated that Hsp90 in tumors is inhibited for between 1 and 5 days. (Banerji et al. 2005).

The patient populations evaluated in the Phase 1 trials conducted to date consist almost exclusively patients with refractory or resistant solid tumors, and only limited clinical activity has been observed. Thus, despite intensive efforts to develop 17-AAG as an anti-cancer agent, 17-AAG has still not been approved by any regulatory authority for use in the treatment of any cancer. There remains a need for methods of dosing and administering 17-AAG and prodrugs of 17-AAG and 17-AG so that the potential therapeutic benefits of the compound can be realized in the treatment of cancer.

In about 20 to 30 percent of the breast cancer cases, the human epidermal growth factor receptor 2 protein (also known as HER2, HER2/neu, ErbB2, Neu, or p185) is overproduced, a condition referred to as HER2 overexpression, HER2-positive or HER2+. This situation can arise from overexpression of a normal complement of the HER2 gene or the presence of extra copies of the gene (amplification). Because HER2-positive breast cancers tend to be more aggressive—they grow faster and are more likely to recur—there has been an emphasis on developing treatments for HER2-positive breast cancer.

### BRIEF SUMMARY OF THE INVENTION

The present invention provides a method for treating breast cancer in a subject in need of such treatment, comprising administering a therapeutically effective dose of 17-AAG or 17-AG or a prodrug of either 17-AAG or 17-AG and a therapeutically effective dose of a HER2 (protein) inhibitor to the subject, and optionally repeating said step until no further therapeutic benefit is obtained.

In one embodiment, the method comprises the administration of multiple doses of 17-AAG or a prodrug thereof to a patient with breast cancer over a time period of at least 4 weeks, wherein each such dose is in the range of about 300 mg/m² to about 450 mg/m² of 17-AAG or an equivalent amount (on a molar basis) of 17-AG or a prodrug of 17-AAG or 17-AG. In one embodiment, the dose is about 375 to about 450 mg/m². In one embodiment, this dose is administered once weekly for at least four weeks. In one embodiment, this dose is administered once weekly for each week in a four week period, which rate of dosing per four week period is called a cycle, and multiple cycles of such treatment are administered to the breast cancer patient. In one embodiment, a complete treatment is no more than 6 cycles.

In one embodiment, the therapeutically effective dose of 17-AAG or a prodrug of 17-AAG is a dose that results in an AUCₜₒₜₐₗ of 17-AAG per dose in the range of about 13,000 ng/mL*h to 48,000 ng/mL*h. In one embodiment, this dose is administered at a rate and frequency such that the Cₘₐₓ of 17-AAG does not exceed 14,000 ng/mL. In one embodiment, this dose is administered at a rate and frequency such that the Cₘₐₓ of 17-AAG is greater than 3,600 ng/mL, preferably greater than 5,000 ng/mL. In one embodiment, this dose is administered at a rate and frequency such that the Cₘₐₓ of 17-AAG is greater than 3,600 ng/mL but less than 14,000 ng/mL, preferably greater than 5,000 ng/mL but less than 14,000 ng/mL.

In one embodiment, the therapeutically effective dose of 17-AG or a prodrug of 17-AG (which prodrug includes 17-AAG) is a dose that results in an AUCₜₒₜₐₗ of 17-AG per dose in the range of about 5,800 ng/mL*h to 39,000 ng/mL*h. In one embodiment, this dose is administered at a rate and frequency such that the Cₘₐₓ of 17-AG does not exceed 3,300 ng/mL. In one embodiment, this dose is administered at a rate and frequency such that the Cₘₐₓ of 17-AG is greater than 800 ng/mL, preferably greater than 1,100 ng/mL. In one embodiment, this dose is administered at a rate and frequency such that the Cₘₐₓ of 17-AG is greater than 800 ng/mL but less than 3,300 ng/mL, preferably greater than 1,100 ng/mL but less than 3,300 ng/mL.

In one embodiment, the therapeutically effective dose of 17-AAG, 17-AG, or a prodrug of either is a dose that results in a combined AUCₜₒₜₐₗ of 17-AAG and 17-AG per dose in the range of about 23,000 ng/mL*h to 82,000 ng/mL*h. In one embodiment, this dose is administered at rate and frequency such that the Cₘₐₓ of 17-AAG does not exceed 14,000 ng/mL and/or the Cₘₐₓ of 17-AG does not exceed 3,300 ng/mL. In one embodiment, this dose is administered at a rate and frequency such that the Cₘₐₓ of 17-AAG is greater than 3,600 ng/mL (preferably greater than 5,000 ng/mL) and/or the Cₘₐₓ of 17-AG is greater than 800 ng/mL (preferably greater than 1,100 ng/mL). In one embodiment, this dose is administered at a rate and frequency such that the Cₘₐₓ of 17-AAG is greater than 3,600 ng/mL but less than 14,000 ng/mL (preferably greater than 5,000 ng/mL but less than 14,000 ng/mL) and/or the Cₘₐₓ of 17-AG is greater than 800 ng/mL but less than 3,300 ng/mL (preferably greater than 1,100 ng/mL but less than 3,300 ng/mL).

In one embodiment, the therapeutically effective dose of 17-AAG or a prodrug of 17-AAG is a dose that results in a Terminal t_{1/2} (h) of 17-AAG in the range of 1.5 to 12. In one embodiment, the therapeutically effective dose of 17-AAG or a prodrug of 17-AAG is a dose that results in a Terminal t_{1/2} (h) of 17-AAG in the foregoing range and an AUCₜₒₜₐₗ of 17-AAG per dose in the range of about 13,000 ng/mL*h to 48,000 ng/mL*h.

In one embodiment, the therapeutically effective dose of 17-AG or a prodrug of 17-AG is a dose that results in a Terminal t_{1/2} (h) of 17-AG in the range of 3.7 to 8.8. In one embodiment, the therapeutically effective dose of 17-AG or a prodrug of 17-AG is a dose that results in a Terminal t_{1/2} (h) of 17-AG in the foregoing range and an AUCₜₒₜₐₗ of 17-AG per dose in the range of about 5,800 ng/mL*h to 39,000 ng/mL*h.

In one embodiment, the therapeutically effective dose of 17-AAG or a prodrug of 17-AAG is a dose that results in a Volume of distribution V_{z} (L) of 17-AAG in the range of 67 to 800. In one embodiment, the therapeutically effective dose of 17-AAG or a prodrug of 17-AAG is a dose that results in a Volume of distribution V_{z} (L) of 17-AAG in the foregoing range and an AUCₜₒₜₐₗ of 17-AAG per dose in the range of about 13,000 ng/mL*h to 48,000 ng/mL*h.

In one embodiment, the therapeutically effective dose of 17-AAG or a prodrug of 17-AAG is a dose that results in a Clearance (L/h) in the range of 13 to 52. In one embodiment, the therapeutically effective dose of 17-AAG or a prodrug of 17-AAG is a dose that results in a Clearance (L/h) of 17-AAG in the foregoing range and an AUCₜₒₜₐₗ of 17-AAG per dose in the range of about 13,000 ng/mL*h to 48,000 ng/mL*h.

In one embodiment, the therapeutically effective dose of 17-AAG or a prodrug of 17-AAG is a dose that results in a Vₛₛ (L) in the range of 66 to 550. In one embodiment, the therapeutically effective dose of 17-AAG or a prodrug of 17-AAG is a dose that results in a Vₛₛ (L) of 17-AAG in the foregoing range and an AUCₜₒₜₐₗ of 17-AAG per dose in the range of about 13,000 ng/mL*h to 48,000 ng/mL*h.

In a preferred embodiment, the subject has HER2-positive breast cancer. In one embodiment, the 17-AAG and a HER2 inhibitor are each administered in separate pharmaceutical formulations. In another embodiment, the 17-AAG and HER2 inhibitor are in the same pharmaceutical formulation. In one embodiment, the HER2 inhibitor is trastuzumab (Herceptin™). In one embodiment, 17-AAG is administered over 120 minutes as an infusion with trastuzumab. In one embodiment, the HER2 inhibitor is trastuzumab and is administered at 4.0 mg/kg over 90 minutes as the loading dose and at 2.0 mg/kg over 30 minutes as the weekly maintenance dose. In one embodiment, the method further comprises testing the subject for HER2 overexpression in the solid tumor prior to the administering step.

### BRIEF DESCRIPTION OF THE DRAWING(S)

FIG. 1 shows the mean and standard deviation (SD) plasma concentration of 17-AAG and 17-AG versus time for a dose of 225 mg/m² 17-AAG for a three-patient cohort.

FIG. 2 shows the mean and SD plasma concentration of 17-AAG and 17-AG versus time for a dose of 300 mg/m² 17-AAG for a three-patient cohort.

FIG. 3 shows mean and SD the plasma concentration of 17-AAG and 17-AG versus time for a dose of 375 mg/m² 17-AAG for an eight-patient cohort.

FIG. 4 shows mean and SD the plasma concentration of 17-AAG and 17-AG versus time for a dose of 450 mg/m² 17-AAG for a ten-patient cohort.

FIG. 5 shows the mean plasma concentration of 17-AAG versus time for doses of 225 mg/m², 300 mg/m², 375 mg/m², and 450 mg/m² of 17-AAG.

FIG. 6 shows the mean plasma concentration of 17-AG versus time for doses of 225 mg/m², 300 mg/m², 375 mg/m², and 450 mg/m² of 17-AAG.

FIG. 7 shows the AUCₜₒₜₐₗ of 17-AAG and 17-AG versus dose.

FIG. 8 shows the total exposure (the sum of AUCₜₒₜₐₗ of 17-AAG and 17-AG) versus dose.

FIG. 9 shows the serum concentration of trastuzumab versus time for 17-AAG doses of 225 mg/m², 300 mg/m², and 375 mg/m².

FIGS. 10A, 10B, 10C, and 10D show the immunoblots for raf-1, AKT, cdk4, Hsp70 , and p85 for four patients in cohort 3. The samples were taken prior to initial infusion (“PreTx”), 6 hours after initial infusion (“6 h”), on Day 2 (“d2”), on Day 3 (“d3”), on Day 8 prior to the Day 8 infusion (“d8”), and on Day 15 prior to the Day 15 infusion (“d15”).

### DETAILED DESCRIPTION OF THE INVENTION

Definitions

To aid in understanding and practice of the present invention, definitions for certain terms used herein are provided below.

A “2+” or “3+” HER2 overexpression means a moderate or intense staining, respectively, of the complete cell membrane in more than 10% of tumor cells of a lesion. Staining of tumor cells is performed using IHC or FISH methodologies as disclosed in Ridolfi et al. 2000.

“17-AAG” is defined to include a prodrug of 17-AAG, and a concentration of 17-AAG is defined to include a molar equivalent concentration of the prodrug of 17-AAG.

“17-AG” is defined to include a prodrug of 17-AG, and a concentration of 17-AG is defined to include a molar equivalent concentration of the prodrug of 17-AG.

An “adverse event” is as defined in National Cancer Institute (2003).

A “dose limiting toxicity” (DLT) is defined as any of the following clinical toxicities, referencing National Cancer Institute (2003). Hematologic toxicities comprise: (1) Grade 4 neutropenia (absolute neutrophil count (ANC) <0.5×10⁹/L) for more than 5 consecutive days, or febrile neutropenia (ANC <1.0×10⁹/L, fever ≧38.5° C.), (2) Grade 4 thrombocytopenia (platelets <25.0×10⁹/L or bleeding episode requiring platelets transfusion), and/or Grade 4 anemia (Hemoglobin <6.5 g/dl). Non-Hematologic toxicities comprise: (1) any ≧Grade 3 non-hematologic toxicity (except Grade 3 injection site reaction, alopecia, anorexia, fatigue), (2) nausea, diarrhea and/or vomiting of Grade ≧3 despite the use of maximal medical intervention and/or prophylaxis, and/or (3) treatment delay of more than 4 weeks due to prolonged recovery from a drug-related toxicity.

“KPS performance status” is defined in Table 1, which also provides a comparison against the ECOG Scale.

TABLE 1
KPS Performance Status

| Karnofsky Scale | | ECOG Scale | |
|---|---|---|---|
| Normal, no complaints | 100 | Fully active, able to carry | 0 |
| | | on all pre-disease perform- | |
| | | ance without restriction | |
| Able to carry on normal | 90 | | |
| activity, minor signs | | | |
| or symptons of disease | | | |
| Normal activity with | 80 | Restricted in physically | 1 |
| effort | | strenuous activity but ambu- | |
| | | latory and able to carry out | |
| | | work of a light or sedentary | |
| | | nature (e.g., office work or | |
| | | light house work) | |
| Unable to carry on | 70 | | |
| normal activity or | | | |
| perform active work; | | | |
| cares for self | | | |
| Requires occasional | 60 | Ambulatory and capable of all | 2 |
| assistance but is able | | self-care but unable to carry | |
| to care for most own | | out any work activities; up | |
| needs | | and about more than 50% of | |
| | | waking hours | |
| Requires considerable | 50 | | |
| assistance and frequent | | | |
| medical care | | | |
| Disabled; requires | 40 | Capable of only limited self- | 3 |
| special medical care | | care, confined to bed or chair | |
| and assistance | | more than 50% of waking hours | |
| Severely disabled; | 30 | | |
| hospitalization | | | |
| indicated although | | | |
| death not imminent | | | |
| Very sick; hospitalized | 20 | Completely disabled; cannot | 4 |
| and active | | perform any self-care; totally | |
| | | confined to bed or chair | |
| Moribund; fatal processes | 10 | | |
| progressing rapidly | | | |
| Dead | 0 | | |

A “measurable lesion” means a lesion that can be accurately measured in at least one dimension as ≧20 mm with conventional techniques or as ≧10 mm with spiral computed tomography (CT) scan. Clinical lesions are considered measurable when they are superficial (e.g., skin nodules, palpable lymph nodes). A “non-measurable lesion” means all lesions other than a “measurable lesion”.

“Tumor response” means the following Response Evaluation Criteria in Solid Tumors (RECIST) criteria (Therasse et al. 2000) for assessment of tumor response and determination of Best Overall Response. Table 2 provides overall responses for all possible combinations of tumor responses in target and non-target lesions with and without the appearance of new lesion(s).

TABLE 2
Overall Response Criteria

| | | | Overall |
|---|---|---|---|
| Target lesions¹ | Non-Target lesions² | New Lesions³ | Response |
| CR | CR | No | CR |
| CR | Incomplete response/StD | No | PR |
| PR | Non-PrD | No | PR |
| StD | Non-PrD | No | StD |
| PrD | Any response | Yes or No | PrD |
| Any response | PrD | Yes or No | PrD |
| Any response | Any response | Yes | PrD |

¹Measurable lesions only.
²May include measurable lesions not followed as target lesions or non-measurable lesions.
³Measureableor non-measurable lesions.

A “complete response” (CR) means, for target lesions, the disappearance of all target lesions. A CR means, for non-target lesions, the disappearance of all non-target lesions and normalization of tumor marker levels.

A “partial response” (PR) means, for target lesions, at least a 30% decrease in the sum of the longest diameter of the target lesions, taking as a reference the baseline sum longest diameter. To be assigned a status of confirmed PR or CR, changes in tumor measurements in patients with responding tumors is confirmed by repeat studies that must be performed ≧4 weeks after the criteria for response are first met.

A “stable disease” (StD) means, for target lesions, neither sufficient shrinkage to qualify as PR nor sufficient increase to qualify as PrD, taking as a reference the smallest sum of the longest diameter since the treatment period. Follow-up measurements must have met the StD criteria at least once after study entry at a minimum interval of 6 weeks.

An “incomplete response/stable disease” (Incomplete Res./StD) means,for non-target lesions, a persistence of one or more non-target lesions and/or maintenance of tumor marker levels above the normal limits. The cytological confirmation of the neoplastic origin of any effusion that appears or worsens during treatment when the measurable tumor has met criteria for response or StD is mandatory to differentiate between response or StD and PrD.

A “progressive disease” (PrD) means, for target lesions, at least 20% increase in the sum of the longest diameter of target lesions, taking as a reference the smallest sum of longest diameter recorded since the treatment started, or the appearance of one or more new lesions. For non-target lesions, a PrD is defined as the appearance of one or more new lesions and/or unequivocal progression of existing non-target lesions.

A “target lesion” means all measurable lesions (up to a maximum of 10) that are representative of all involved organs. Target lesions are measured and recorded at baseline and at the stipulated intervals during treatment. Target lesions are selected on the basis of their size (lesions with the longest diameters) and their suitability for accurate repetitive measurements (either by imaging techniques or clinically). The longest diameter is recorded for each target lesion. The sum of the longest diameter for all target lesions is calculated and is used as the baseline reference to further characterize the objective tumor response to treatment. A “non-target lesion” is any lesion that is not a target lesion.

A “therapeutically effective dose” means, unless otherwise indicated, the amount of drug that is required to be administered to achieve the desired therapeutic result.

A “tumor marker response” means a reduction by ≧50% in the value of the tumor marker relative to baseline.

A “tumor marker progression” means the occurrence of either of the following: (1) relative to pretreatment measurements: an increase in the tumor marker by >25% from a pretreatment marker level of >200 Units, or an increase by >50% from a pretreatment marker level of ≦200 Units, or (2) relative to the measurements at the lowest on-study marker level (the “nadir on-study marker level”): an increase in the tumor marker by >25% from a nadir on-study marker level of >200 Units, or an increase by >50% from a nadir on-study marker level of ≦200 Units.

### EMBODIMENTS

The present invention provides important new methods for treating breast cancer (especially HER2-positive breast cancer) using 17-AAG, 17-AG, and prodrugs of 17-AAG or 17-AG, in combination with a HER2 inhibitor. The present invention arose in part from the discovery of new methods for dosing and administering 17-AAG to achieve and maintain therapeutically effective blood levels of 17-AAG or 17-AG (or blood levels of 17-AAG added together with 17-AG, as these moieties are equipotent in cellular assays), expressed as AUCₜₒₜₐₗ, Cₘₐₓ, Terminal t_{1/2}, Clearance, and Volume of distribution, expressed as either V_{z} or Vₛₛ, without reaching blood levels that cause unmanageable toxicity in breast cancer patients as well as the discovery that trastuzumab and other HER2 inhibitors can potentiate the anti-cancer activity of these compounds in breast cancer patients.

In one embodiment, the HER2 inhibitor is administered prior to administering 17-AAG or 17-AG or a prodrug of either. In another embodiment, 17-AAG or 17-AG or a prodrug of either is administered prior to administering the HER2 inhibitor. In yet another embodiment, the two types of drugs are administered concomitantly. In one embodiment, the 17-AAG or 17-AG or a prodrug of either and the HER2 inhibitor are administered separately during a one week period.

In one embodiment, the invention comprises administering multiple doses of 17-AAG, 17-AG, or a prodrug of either, in combination with a HER2 inhibitor, over a period of four weeks. Collectively, these four doses over the four week period are called a cycle of treatment or, simply, a cycle. A patient may be treated with multiple cycles. Different cycles, including cycles of longer or shorter duration or involving greater or fewer doses than described specifically herein, can be used, so long as the therapeutically effective amounts and pharmacokinetic parameters described herein are achieved.

In one embodiment, the therapeutically effective dose is achieved by the administration of multiple doses of 17-AAG, 17-AG, or a prodrug of 17-AAG or 17-AG, in combination with (including separate administration within at least one week of one another) a HER2 inhibitor, to a patient with breast cancer over a time period of at least 4 weeks, wherein such multiple doses result in an AUCₜₒₜₐₗ for 17-AAG per dose of at least 13,000 ng/mL*h but less than 48,000 ng/mL*h. In one embodiment, four doses are administered per cycle, with each dose being at least 300 mg/m², with a period of 1 week between each dose.

Compounds other than 17-AAG or 17-AG can be administered, if they are converted in vivo to 17-AAG or 17-AG (i.e., prodrugs of 17-AAG or 17-AG). One type of prodrug is that in which the geldanamycin benzoquinone ring is reduced to a hydroquinone ring, but is metabolized back to a benzoquinone ring in the subject, specific example of a 17-AAG prodrug being 17-allylamino-18,21-dihydro-17-demethoxygeldanamycin (17-AAGH₂). Adams et al. 2005a and 2005b. As 17-AAG is in turn converted in vivo to 17-AG (Egorin et al. 1998) and 17-AG has activity approximately equal to that of 17-AAG (Schnur et al. 1995a and 1995b), 17-AAGH₂ can be considered to be also a prodrug of 17-AG. As 17-AAGH₂ in its free base form air oxidizes readily, it is preferably handled as it ammonium salt or as a solution formulation comprising an antioxidant (e.g., ascorbic acid), a low-pH buffering agent (e.g., citric acid/citrate), and a metal chelator (e.g., EDTA).

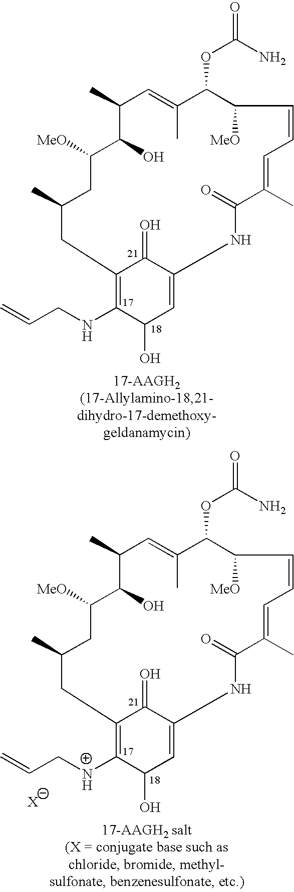

The methods of the invention include, in one embodiment, a method for treating breast cancer in a patient in need of said treatment, wherein the method comprises the administration of multiple doses of 17-AAG or 17-AG, or a prodrug of 17-AAG or 17-AG, such as 17-AAGH₂, to a patient with breast cancer, over a time period of at least 4 weeks, wherein such multiple doses result in an AUCₜₒₜₐₗ for 17-AG per dose of at least 5,800 ng/mL*h but less than 39,000 ng/mL*h. In one embodiment, four doses are administered per cycle, with each dose being at least 300 mg/m², and a period of 1 week between each dose.

Thus, in the method of treatment of the present invention, the term “administering” encompasses the treatment of breast cancer with a compound that converts to 17-AAG or 17-AG in vivo after or concurrently with administration to the subject. In the specific case of 17-AAGH₂, “administrating encompasses administering its salt or a solution thereof. Other 17-AAG or 17-AG prodrugs can be used, conventional procedures for the selection and preparation of which are described, for example, in Wermuth 2003.

A HER2 inhibitor is a molecule that (1) is capable of inhibiting HER2 by blocking or reducing the downstream signaling through the mitogen-activated protein kinase (MAPK) and/or Akt/phosphoinositide 3-kinase (PI3-kinase) pathways and/or (2) exerts its therapeutic action by a mechanism substantially similar to that of trastuzumab. The mechanism of action of trastuzumab is believed to involve (a) inhibition of the HER2 function (e.g., by binding to the HER2 extracellular domain thus preventing binding by its cognate ligand, by binding to the cognate ligand and inhibiting its binding to HER2, by downregulating HER2, or by inhibiting the tyrosine kinase activity of HER2) and/or (b) binding to the extracellular domain of HER2 in a tumor cell and marking the cell for attack by the host's immune system. Additionally, trastuzumab may sensitive an otherwise insensitive or resistant tumor cell to cytotoxic factors such as tumor necrosis factor a (TNF-α). For a discussion of the possible mechanisms of action of trastuzumab, see, e.g., Hudziak et al. 1997 and Genentech 2005. Inhibition can result in delayed tumor growth, induced tumor shrinkage, enhanced cytotoxic chemotherapy, and reduced metastases (De Bono and Rowinsky, 2002). In one embodiment, the HER2 inhibitor is a small organic molecule, a peptide or peptide mimetic, or an antibody, or any functional fragment or antibody fragment thereof that binds to HER2. Examples of such small organic molecules are pyrimido-pyrimidines (Hilberg et al. 2003), quinazolines (Tang et al. 2000a and 2000b), indazolylpyrrolotriazines (Vite et al. 2005), arylindolinones (Cui et al. 2004), and lapatinib (Carter et al. 2004; Xia et al. 2005). Examples of peptides or peptide mimetic inhibitors are disclosed in Greene et al. 2003; Park et al. 2000.

In one embodiment, the HER2 inhibitor is an antibody, or any functional fragment or antibody fragment thereof, that binds to HER2 and thereby blocks or reduces the downstream signaling through the MAPK and/or Akt/PI3-kinase pathways. The range of anti-HER2 antibodies encompassed by this invention encompasses the compounds defined in Hudziak et al. 1997, 1998a, 1998b, 2000, 2002a, and 2002b. Examples of anti-HER2 antibodies are the monoclonal antibodies 4D5, 3E8, 3H4, and trastuzumab. In another embodiment, the HER2 inhibitor is an antibody, or any functional fragment or antibody fragment thereof, that binds to the antigen bound by 4D5 or trastuzumab. In another embodiment, the HER2 inhibitor is an antibody, or any functional fragment or antibody fragment thereof, which comprises the complementarity-determining regions (CDR) of 4D5 or trastuzumab. In one embodiment, the monoclonal anti-HER2 antibody is a humanized monoclonal antibody. In one embodiment, the monoclonal anti-HER2 antibody is trastuzumab.

Trastuzumab (Herceptin®, Genentech, South San Francisco, Calif., USA) is a humanized monoclonal antibody that binds to HER2. Methods for making and using of trastuzumab and suitable pharmaceutical formulations and means and modes of administration thereof are taught in Hudziak et al. 1997, 1998a, 1998b, 2000, 2002a, and 2002b. Trastuzumab is approved as a single agent for (1) the treatment of patients with metastatic breast cancer whose tumors overexpress the HER2 protein and who have received one or more chemotherapy regimens for their metastatic disease, and (2) the treatment, in combination with paclitaxel, of patients with metastatic breast cancer whose tumors overexpress the HER2 protein and who have not received chemotherapy for their metastatic disease. In one embodiment, trastuzumab is used in patients whose tumors have been evaluated with an assay validated to predict HER2 overexpression. Methods of assaying for HER2 protein overexpression include methods that utilize immunohistochemistry (IHC) and methods that utilize fluorescence in situ hybridization (FISH). A commercially available IHC test is PathVysion® (Vysis Inc., Downers Grove, Ill.). A commercially available FISH test is DAKO HercepTest® (DAKO Corp., Carpinteria, Calif.).

When used in vivo for therapy, the anti-HER2 antibodies are administered in therapeutically effective amounts. Preferably, they are administered parenterally, when possible, at the target cell site, or intravenously (IV). The amount of anti-HER2 antibody administered will typically be in the range of about 0.1 to about 10 mg/kg of patient weight. For parenteral administration, the anti-HER2 antibodies are formulated in a unit dosage injectable form (solution, suspension, emulsion) in association with a pharmaceutically acceptable parenteral vehicle. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The anti-HER2 antibodies are typically formulated in such vehicles at concentrations of about 1 mg/ml to 10 mg/ml. When the anti-HER2 antibody is trastuzumab, the trastuzumab is preferably administered in a pharmaceutical formulation comprising about 440 mg trastuzumab, about 400 mg α,α-trehalose dihydrate, about 9.9 mg L-histidine, and about 1.8 mg polysorbate 20, which has been reconstituted with about 20 mL of bacteriostatic water for injection (BWFI) or sterile water for injection (SWFI). In a preferred embodiment, the pharmaceutical formulation comprising trastuzumab comprises about 21 mg/mL trastuzumab and has a pH of about 6. In one embodiment, each dose of the trastuzumab administered is from 1 to 4 mg/kg, preferably about 2 to 4 mg/kg. In a more preferred embodiment, the initial or loading dose of trastuzumab is about 4 mg/kg and all subsequent or maintenance doses of trastuzumab are about 2 mg/kg. Typically, the dose is administered as an IV infusion.

In another embodiment, the HER2 inhibitor is a dual tyrosine kinase inhibitor, that is, an inhibitor that inhibits not just HER2 but also a second tyrosine kinase, typically EGFR (also known as ErbBl). Examples of dual tyrosine kinase inhibitors include Gefitinib (Iressa®), Erlotinib (Tarceva®) and lapitinib. de Bono and Rowinsky 2002; Johnston 2006.

The subject in need of treatment, for purposes of the present invention, is typically a human patient suffering from breast cancer, although the methods of the invention can be practiced for veterinary purposes, with suitable adjustment of the unit dose to achieve the equivalent AUCₜₒₜₐₗ or other PK and PD parameters described herein for the particular mammal of interest (including cats, cattle, dogs, horses, and the like). Those of skill in the art of pharmaceutical science know or can readily determine the applicable conversion factors for the species of interest from the present disclosure of the doses and PK parameters for human therapy.

In one embodiment, the subject has been diagnosed with a histologically confirmed breast cancer malignancy. In one aspect, the subject has metastatic breast cancer with 2+ or 3+ HER2 overexpression. In one embodiment, the subject has progression of disease following treatment with trastuzumab. When the HER2 inhibitor is trastuzumab, the subject preferably is not pulmonary compromised, does not have a pre-existing cardiac dysfunction, and/or is not hypersensitive to any Chinese Hamster Ovary protein.

A therapeutically effective dose of 17-AAG and a therapeutically effective dose of a HER2 inhibitor are the amounts of 17-AAG and HER2 inhibitor, respectively, that are administered at each administration over one treatment cycle to the subject that brings about a therapeutic result. The therapeutic result can be that the rate of the progression or spread of the cancer is slowed or stopped for some period of time. In some patients, the therapeutic result can be partial or complete elimination of a target or non-target lesion. A therapeutic result can be achieved with one or multiple treatment cycles. However, there can be no assurance that every breast cancer patient will achieve a therapeutic result with any anti-cancer therapy.

As noted above, a treatment cycle can be four weeks. In other embodiments, the weekly dose can be employed for any suitable number of weeks, so long as the equivalent AUCₜₒₜₐₗ or other PK and PD parameters described herein are achieved. The unit dose employed in each cycle is administered at least once per week.

Each unit dose of 17-AAG is a dose of not more than the maximally tolerable dose (MTD). The MTD can be defined as the maximum dose at which none or only one of six subjects undergoing the method of treatment experiences hematologic or non-hematologic toxicity not amenable to supportive care. Preferably, the amount of 17-AAG administered is equal to or less than the MTD. Preferably, the amount of 17-AAG administered is one that does not result in unacceptable and/or unmanageable hematologic or non-hematologic toxicity. In one embodiment, the MTD is 450 mg/m²/dose.

In one embodiment, the amount of 17-AAG administered in a single unit dose can range from 300 mg/m²/dose to 450 mg/m²/dose. In another embodiment, the amount of 17-AAG administered in a single unit dose is about 225 mg/m²/dose; 300 mg/m²/dose; 375 mg/m²/dose; and 450 mg/m²/dose. Where the 17-AAG is administered once weekly for four weeks, the amount of 17-AAG administered ranges from 225 to 450 mg/m²/dose. Where the 17-AAG is administered once weekly, the amount of 17-AAG administered ranges from 300 to 450 mg/m²/dose. Where the 17-AAG is administered once weekly, the amount of 17-AAG administered ranges from 375 to 450 mg/m²/dose. In another embodiment of once weekly administration, the amount of 17-AAG administered is 450 mg/m²/dose. Those of skill in the art will recognize that the unit dose amounts of 17-AAG or 17-AG prodrugs or 17-AG itself can be calculated from the doses provided herein for 17-AAG and the PK parameters provided for 17-AAG and 17-AG and the molecular weight and relative bioavailability of the prodrug or 17-AG metabolite.

The invention can also be described in terms of the amount of 17-AAG administered per treatment cycle. The per-cycle amount will typically be equal to or greater than 1,200 mg/m², and more usually will be equal to or greater than 1,500 mg/m². The amount of 17-AAG administered can be at least 1,200 to 1,800 mg/m²/treatment cycle; and 1,500 to 1,800 mg/m²/treatment cycle.

As noted above, the frequency of the administration of the unit dose is once weekly or twice weekly. In one embodiment, the pharmaceutical formulation is administered intravenously once weekly for 3 or 4 weeks of a four week period. Administration can be on the same day of the week for every week. The patient can be administered a pre-treatment medication to prevent or ameliorate treatment related toxicities. Illustrative pre-treatment medications are described in the examples below. 17-AAG and the HER2 inhibitor are typically administered by IV infusion, infused in a period of at least 30, 60, 90, or 120 minutes. For patients with a BSA greater than 2.4 m², dosing can be calculated in accordance with the methods herein using a maximum BSA of 2.4 m².

In human clinical trials, an administration regimen of 450 mg/m²/single administration of 17-AAG once weekly for four weeks has been employed without reaching DLT in any treated patient.

Those skilled in the art will understand that, in the foregoing paragraphs the dosages have been expressed in terms of 17-AAG for the sake of conciseness, but that a molar equivalent amount of 17-AG or a prodrug of 17-AAG or 17-AG, or combinations of 17-AAG, 17-AG or a prodrug of 17-AAG or 17-AG can be used instead.

As noted above, after 17-AAG is administered, the major metabolite of 17-AG, having anticancer activity in its own right, appears in the subject. 17-AAG and 17-AG are thus each, and together, responsible for the therapeutic benefit of the method of the invention. The therapeutically effective dose and dosing regimen of 17-AAG is one that achieves an Area Under Curve (AUCₜₒₜₐₗ) of 17-AAG and/or 17-AG in the subject as described herein. Various therapeutically effective doses and dosing regimen are illustrated in the examples below. Therapeutically effective doses and dosing regimen of 17-AAG and/or 17-AG provided by the present invention can also be described in terms of Terminal Half Life (t_{1/2}); Clearance (CL); and Volume of Distribution in the elimination phase or steady state (V_{z} and/or Vₛₛ).

The therapeutically effective amount of a unit dose can be an amount that, after one or more cycles of administration in accordance with the method of the invention, results in a therapeutic benefit. The therapeutic benefit from the treatment method of the present invention can be observed in responding subjects as soon as 4, 8, 12, 16, 20, or 24 weeks from the start of treatment (first administration of the pharmaceutical formulation). The therapeutic benefit can be a CR, PR, or StD (or incomplete response/StD) as defined in this specification for target lesions, non-target lesions, or overall response. Some patients will not relapse from a CR or will experience a significant delay in the progression of the disease or will experience no further metastasis of their cancer. Another therapeutic benefit can be an improvement of the KPS of the patient by 10% or more, 20% or more, 30% or more, 40% or more, or 50% or more. Another therapeutic benefit can be an improvement of the ECOG of the patient by 1, 2, 3 or more.

The present invention provides, in various embodiments, methods for treating breast cancer by administering 17-AAG or 17-AG or a prodrug of either in combination with a HER2 inhibitor and yet another anti-cancer compound (such as doxorubicin, cyclophosphamide, epirubicin, vinorelbine, paclitaxel, docetaxel, capecitabine, gemcitabine, tamoxifen, fulvestrant, a platinum drug, etoposide, vinblastine, or fluorouracil).

Importantly, the the present invention can be used to treat patients with breast cancer who have failed one or more prior anti-cancer therapy regimens. These prior anti-cancer regimens include, but are not limited to, monotherapy, combination therapy, surgery, and radiation therapy. In one important embodiment, the method is applied to the treatment of patients who have cancers that have proven resistant to Herceptin monotherapy or a Herceptin combination therapy with another chemotherapy agent.

An active pharmaceutical ingredient (“API,” 17-AAG, 17-AG, a prodrug of either, HER2 inhibitor, other anti-cancer compound, etc.) useful in the method of the present invention can be formulated for administration orally or intravenously, in a suitable solid or liquid form. See Gennaro, ed., Remington: The Science and Practice of Pharmacy, 20th Ed. (Lippincott Williams & Wilkins 2003), incorporated herein by reference. The API can be compounded, for example, with a non-toxic, pharmaceutically acceptable carrier or excipient for solutions, emulsions, suspensions, or any other form suitable for enteral or parenteral administration. Pharmaceutically acceptable carriers include water and other carriers suitable for use in manufacturing preparations in liquefied form. In addition, auxiliary stabilizing, thickening, and coloring agents may be used.

An API useful in the method of the invention may be formulated as micro-capsules, nanoparticles, or nanosuspensions. General protocols for such formulations are described, for example, in Microcapsules and Nanoparticles in Medicine and Pharmacy by Max Donbrow, ed., CRC Press (1992) and in Bosch et al. 1996; De Castro 1996, and Bagchi et al. 1997. By increasing the ratio of surface area to volume, these formulations are especially suitable for the delivery of 17-AAG or another relatively insoluble API.

17-AAG can be formulated in an emulsion with vitamin E or a PEGylated derivative thereof. Generic approaches to formulations with such excipients are described in Quay et al. 1998 and Lambert et al. 2000. The 17-AAG can be dissolved in an aqueous solution containing ethanol (preferably less than 1% w/v). Vitamin E or a PEGylated-vitamin E is added. The ethanol is then removed to form a pre-emulsion that can be formulated for intravenous or oral routes of administration.

Another method for preparing a pharmaceutical formulation useful in the present method involves encapsulating 17-AAG or other API in liposomes. Methods for forming liposomes as drug delivery vehicles are well known in the art. Suitable protocols adaptable for the present invention include those described by Boni et al. 1997, Straubinder et al. 1995, and Rahman et al. 1995 for paclitaxel and by Sonntag et al. 2001 for epothilone, mutatis mutandis. Of the various lipids that may be used in such formulations, phosphatidylcholine and polyethyleneglycol-derivatized distearyl phosphatidyl-ethanoloamine are noteworthy.

The amount of 17-AAG or other API that may be combined with the carrier materials to produce a single or unit dosage form will vary depending upon the subject treated and the particular mode of administration. For example, a formulation for IV use comprises an amount of 17-AAG ranging from about 1 mg/mL to about 25 mg/mL, preferably from about 5 mg/mL, and more preferably about 10 mg/mL. IV formulations are typically diluted between about 2 fold and about 30 fold with SWFI, normal saline, or 5% dextrose solution prior to use. In many instances, the dilution is between about 5 and about 10 fold.

In one embodiment of the method of the invention, 17-AAG is formulated as a pharmaceutical solution formulation comprising 17-AAG dissolved in a vehicle comprising (i) a first component that is ethanol; (ii) a second component that is a polyethoxylated castor oil; and (iii) a third component selected propylene glycol, PEG 300, PEG 400, glycerol, and combinations thereof, as disclosed in Zhong et al. 2005.

Another formulation of 17-AAG that may be used is one based on dimethylsulfoxide (“DMSO”) and egg lecithin (egg phospholipids), as taught in Tabibi et al. 2004. However, because of certain characteristics of DMSO (odor, patient adverse reactions), such formulations are less preferred than the DMSO-free ones taught herein.

Other formulations for 17-AAG that may be employed in the method of the invention are described in Ulm et al. 2003, Ulm et al. 2004, Mansfield et al. 2006, Desai et al. 2006, and Isaacs et al. 2006.

In another embodiment, the pharmaceutical formulation can be diluted 1:7 prior to administration with sterile WFI, USP (one part undiluted drug product to 6 parts SWFI). Dilution is performed under controlled, aseptic conditions. The final diluted drug product concentration is, using 17-AAG as an example, at least 1.00 mg/mL, such as approximately 1.43, approximately 2.00 or approximately 10.00 mg/mL.

Where the pharmaceutical formulation comprises an additional compound that might cause an anaphylactic reaction (like Cremophor®), additional medications can be administered to prevent or reduce the anaphylactic reaction, such as (a) loratidine or diphenhydramine, (b) famotidine, and (c) methylprednisone or dexamethasone.

Depending on the body surface area and the assigned dose for individual patients, the dose of 17-AAG or other API will require different volumes of drug product to be added to the admixture bag. An overfill can be calculated and employed to account for loss in the administration set. Preferably, the pharmaceutical formulation, with the diluted drug product, is pH neutral, and the solution is hypertonic at approximately 600 mOsm. In one embodiment, the pharmaceutical formulation is stored at −20° C., and is protected from light. Drug product is allowed to come to room temperature prior to admixture. After coming to room temperature, mixing is by gentle inversion. After dilution, the drug product should stable for up to about 10 hours at room temperature (at a dilution of 1:7).

The present invention, having been described in summary fashion and in detail above, is illustrated in the following Example.

Treatment of Breast Cancer Patients with 17-AAG and Trastuzumab

The invention was tested in an open-label, dose escalating clinical trial. The trial was designed to establish the MTD of 17-AAG administered in combination with trastuzumab in patients with advanced solid tumor malignancies (phase 1). The patients had a histologically confirmed malignancy that was metastatic or unresectable and for which standard curative or palliative measures did not exist or were no longer effective. Metastatic disease, if present, was not progressing so as to required palliative treatment. 17-AAG was administered by IV infusion over 120 minutes weekly. Patients were assessed in 4-week cycles. The dose of 17-AAG was escalated starting from 225 mg/m² until a MTD was ascertained.

Disease response evaluations were performed following every two cycles of treatment (approximately 8 weeks). The determination of anti-tumor efficacy in stable or responding patients was based on objective tumor assessments made according to RECIST. Therasse et al. 2000.

The patients enrolled in this study satisfied the following inclusion criteria: (1) age ≧18 years; (2) KPS performance status of ≧70%; (3) histologically confirmed solid tumor malignancy (assessed within 28 days prior to treatment; for the Phase 1 portion of the trial); (4) metastatic breast cancer with 2+ or 3+ HER2 overexpression, with progressive disease following initial treatment for metastatic disease with trastuzumab (as a single-agent or in a combination therapy; for the Phase 2 portion of the trial); (5) all adverse events of any prior chemotherapy, surgery, or radiotherapy were resolved to NCI CTCAE (v. 3.0) Grade ≦2; (6) the following laboratory results, within 10 days of 17-AAG administration: hemoglobin ≧8.5 g/dL, absolute neutrophils count ≧1.5×10⁹/L, platelet count ≧75×10⁹/L, serum bilirubin ≦2× ULN, AST and ALT ≦2× ULN, and serum creatinine ≦2× ULN.

Patients with any of the following attributes were excluded from participation in the study: (1) documented hypersensitivity reaction CTCAE Grade ≧3 to prior therapy containing Cremophor® or trastuzumab; (2) pregnant or breast-feeding; (3) known central nervous system (CNS) metastases; (4) administration of chemotherapy, biological, immunotherapy or investigational agent (therapeutic or diagnostic) within 21 days prior to start of treatment; (5) severe dyspnea at rest; or New York Heart Association (NYHA) class III or IV congestive heart failure, or a left ventricular ejection fraction (LVEF) less than 50%; (6) any medical conditions imposing excessive risk to the patient (such as congestive heart failure of Class III or IV (NYHA classification), infection requiring anti-infective treatment, etc.), and (7) patients with previous malignancies unless free of recurrence for at least 5 years except cured basal cell carcinoma of the skin, carcinoma-in-situ of either the uterine cervix or urinary bladder, or Stage T1 or T2 prostate cancer whose PSA was <2 ng/mL.

PK assessment. PK sampling was obtained during the first treatment cycle only. Blood samples (approximately 55 mL total) for determination of plasma concentrations of 17-AAG and 17-AG were collected following the first 17-AAG administration only (Days 1, 2 and 3). Plasma concentrations were determined by a validated liquid chromatography-mass spectrometry (LC-MS) method. Blood samples for determination of plasma concentrations of trastuzumab were collected following the first infusion only, with serial sampling performed on Day 1, and single samples obtained on Days 2, 3, 8 and 15. Plasma concentrations of trastuzumab were determined using an enzyme-linked immunosorbent assay with a 150 ng/mL limit of quantification.

PD assessment. PD sampling was obtained during the first treatment cycle only. The occurrence of specific toxicities of interest (e.g., severity, duration and reversibility) was compared to pharmacokinetic parameters (e.g., clearance, exposure, elimination half-life, maximal plasma concentration, and time above a target plasma concentration). These toxicities may include hepatotoxicity and gastrointestinal toxicities. The laboratory correlated assessment of Hsp70 and Hsp90-dependent client proteins in peripheral blood lymphocytes. These correlative studies allowed (a) assessment of the degree to which 17-AAG had inhibited Hsp90 function in lymphocytes from patients treated on protocol; and (b) correlation of clinical responses to 17-AAG with the degree of modulation of the biomarkers.

End of treatment assessment. The planned treatment period was 24 weeks (6 cycles). All patients who received at least one dose of the study drug were to have an end-of-treatment assessment, taking place up to 28 days following the last dose of 17-AAG and including a physical examination (with body weight and vital signs measurements), echocardiogram/MUGA, documentation of KPS Performance Status, hematology, coagulation, tumor markers and chemistry/electrolyte determinations, urinalysis, assessment of the patient's current medications and ongoing clinical adverse events (if any). Tumor assessments and echocardiogram/MUGA were to be done only if the previous assessment occurred more than 4 weeks prior to withdrawal.

Administration and schedule. Trastuzumab was administered weekly, initially as a loading dose (4 mg/kg) over 90 minutes; subsequent weekly infusions (2 mg/kg) were administered over 30 minutes, as tolerated. 17-AAG infusions immediately followed the trastuzumab infusions, generally with a lapse of no more than a few minutes. In the Dose Escalating Phase of the study, 17-AAG was administered intravenously weekly at escalating doses (calculated mg/m²) infused over 120 minutes after pre-medication. For patients with a body surface area greater than 2.4 m², dosing was calculated using a maximum BSA of 2.4 m². After determination of a MTD and recommended phase 2 dose for 17-AAG, all subsequent patients are to receive this dose over 120 minutes.

Preparation of 17-AAG. 17-AAG was dissolved in 30% propylene glycol, 20% Cremophor® EL, and 50% ethanol to a concentration of 10 mg/mL in the vial. Drug product was available in 20 mL type 1 clear glass vials with a 20 mm finish (containing 200 mg/vial). The vials were closed with gray 20 mm Teflon coated serum stoppers and white 20 mm flip-off white lacquered flip tops. It was diluted 1:7 prior to administration with SWFI, USP (one part undiluted drug product to 6 parts SWFI). Dilution was performed under controlled, aseptic conditions. Final diluted drug product had a concentration of approximately 1.43 mg/mL. 17-AAG was prepared either using glass vacuum containers or compatible non-PVC, non-DEHP (di(2-ethylhexyl)phthalate) IV admixture bags. Both systems require non-PVC, non-DEHP containing administration sets and either an in-line 0.22 μm filter or use of an extension set containing such a filter. Due to the light sensitivity of 17-AAG, protection from light is advised.

Final diluted drug product had a concentration of approximately 1.43 mg/mL. The 17-AAG was either prepared using glass vacuum containers or compatible non-PVC, non-DEHP IV admixture bags. Both systems required non-PVC, non-DEHP containing administration sets and an in-line, or an extension set containing, a 0.22 μm filter.

Pre-medication treatments. All patients were pre-medicated prior to each infusion of 17-AAG. An appropriate pre-medication regimen for each patient was based upon past history of potential Cremophor®-induced hypersensitivity reactions and the type and severity of the hypersensitivity reaction observed following treatment with 17-AAG. The standard premedication regimen was to pre-medicate with loratidine 10 mg p.o., famotidine 20 mg p.o., and either methylprednisolone 40-80 mg IV or dexamethasone 10-20 mg IV 30 minutes prior to infusion of 17-AAG. The high dose premedication regimen was to pre-medicate with diphenhydramine 50 mg IV, famotidine 20 mg IV and either methylprednisolone 80 mg IV or dexamethasone 20 mg IV (or split as oral doses of 10 mg each 6 and 12 hours prior to the infusion), at least 30 minutes prior to the infusion of 17-AAG.

Dosing. The initial patient cohort received the IV infusion of 17-AAG at a dose of 225 mg/m². Patient cohorts were enrolled per the following escalation scheme: cohort 1 (225 mg/m²); cohort 2 (300 mg/m²); cohort 3 (375 mg/m²); and cohort 4 (450 mg/m²). Trastuzumab was administered at the following doses: 4 mg/kg loading dose during week 1 and then 2 mg/kg weekly ×3 every 4 weeks thereafter. Three patients were assigned to each cohort. If no DLT was observed in a cohort of three patients evaluable for dose escalating decision (“evaluable” is defined here as having received three treatments in a 4-week period or having withdrawn due to drug-related toxicity), then the next dose level was evaluated. If one out of three patients experienced a DLT, then the cohort was increased to six evaluable patients. If two or more of six evaluable patients entered in a cohort experienced a DLT then the MTD was exceeded; if the previous dose level produced DLT in no more than 1 out of 6 patients, then this dose was considered the MTD and was used for all subsequent patients entered in the phase 2 portion of the study.

Twenty-five patients (21 female, 4 male) were treated in accordance with this protocol. Of the female patients, eighteen were diagnosed with Her2 positive metatastic breast cancer (MBC). Other cancers diagnosed were one each with colorectal, kidney, ovarian, uterine and thymic cancers, and two with prostate cancers. The median age of the patients were 66 years old (with a range of 33 to 87 years old). The median KPS was 90 (with a range of 80 to 100). The median time from diagnosis was 60 months (with a range of 13 to 229 months). Of the eighteen MBC patients, the median number of prior treatments was 3 (with a range of 0 to 9; not including endocrine treatments) and the median number of prior trastuzumab-containing regimens was 2 (with a range of 0-5). Of the other seven patients, the median number of prior treatments was 2 (with a range of 0 to 7).

Plasma samples from some of the patients were assayed using a validated good laboratory practice (GLP) compliant analytical method. All plasma samples were analyzed for 17-AAG and 17-AG. The lower limit of quantitation for 17-AAG and 17-AG was 10.0 ng/mL and 5.0 ng/mL, respectively.

Four cohort 1 patients (Patients 102-105) received 17-AAG (225 mg/m²) and trastuzumab. The mean number of cycles administered was 1.8. No DLT was observed.

Three cohort 2 patients (Patients 201-203) received 17-AAG (300 mg/m²) and trastuzumab. The mean number of cycles administered was 6.7. No DLT was observed. One patient from cohort 2 (patient 202) was observed to have a PR following treatment.

Patient 202 (70+ year old female) was diagnosed with Her2+ MBC with active sites of disease (including bone, heart, right kidney, and a left adrenal metastasis). She had a slowly progressive disease on treatment of trastuzumab monotherapy prior to enrollment in this study. She received 8 cycles of treatment (27 infusions) before withdrawal from the study for idiopathic thrombocytopenia.

Eight cohort 3 patients (Patients 301-308) received 17-AAG (375 mg/m²) and trastuzumab. The mean number of cycles administered was 4.3. One patient was observed to have fatigue and abdominal pain. One patient from cohort 3 (patient 306) was observed to have a PR following treatment. Patient 306 (40+ year old female), a patient diagnosed with Her2+ MBC with active sites of diseases (including lung and bone), received 13 infusions before withdrawal from study for hypersensitivity reaction.

Ten cohort 4 patients (Patients 401-410) received 17-AAG (450 mg/m²) and trastuzumab. One patient was observed to have a Grade 4 decrease of platelets. The trastuzumab dose was administered prior to administering 17-AAG.

Overall, there was no observation of bone marrow suppression or cardiovascular toxicity. Only minimal hepatotoxicity was observed.

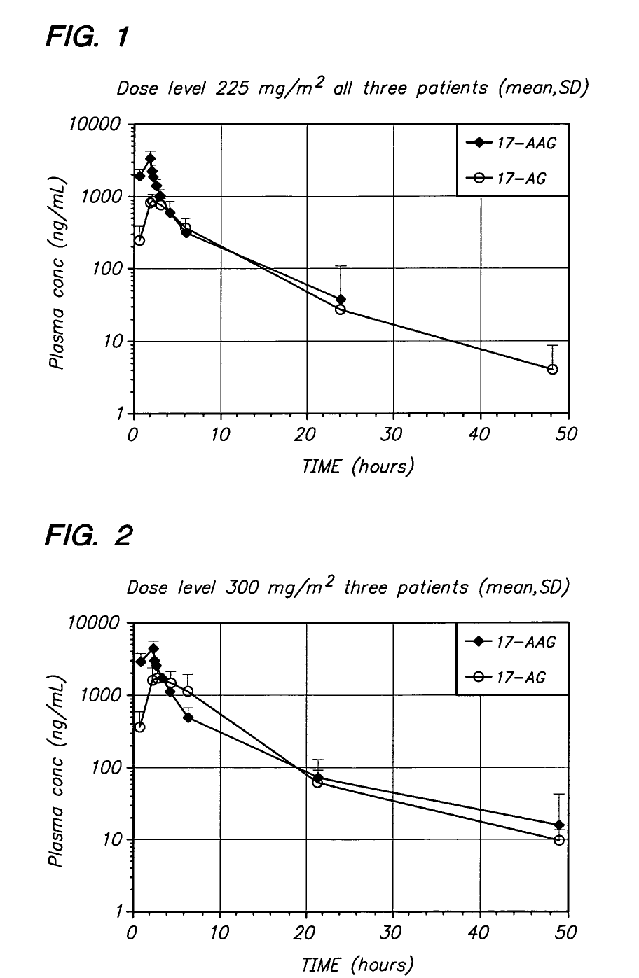

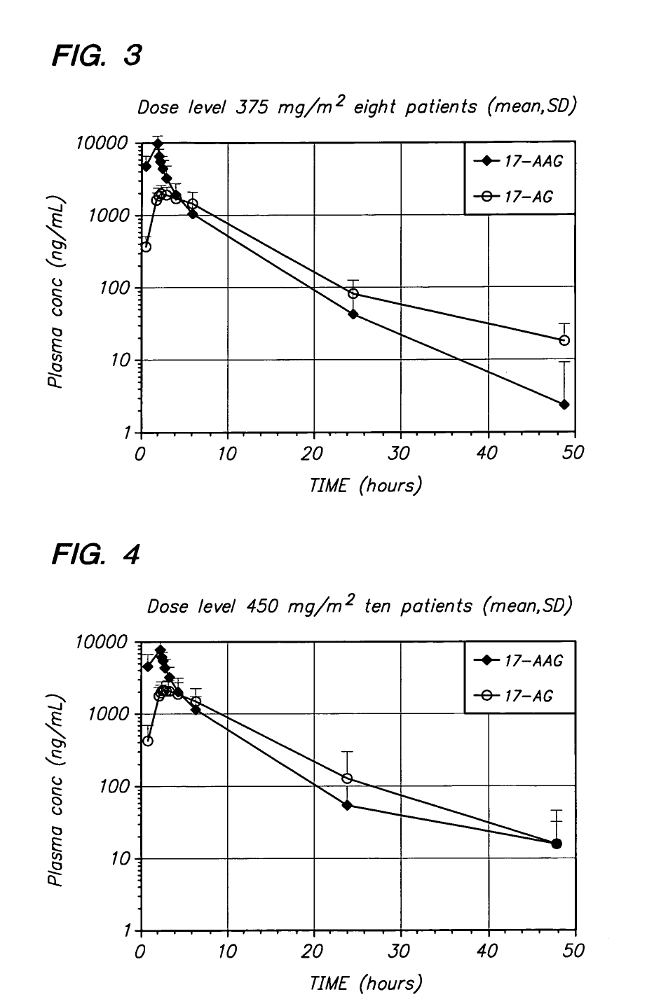

Blood was collected as follows for plasma drug concentration analysis: Pre-dose, 30 minutes intra-infusion, and just before the end-of-infusion (EOI) at 5, 15, 30 minutes and 1, 2, 4, 8, 24, and 48 hours post infusion. The plasma concentrations of 17-AAG and 17-AG were measured for each sample, and the standard PK values were determined. All of the patients' plasma samples were analyzed. FIGS. 1 to 4 show the plasma concentration: time curves for 17-AAG and 17-AG for cohorts 1-4 (17-AAG dose levels 225, 300, 375, and 450 mg/m²) (mean, SD).

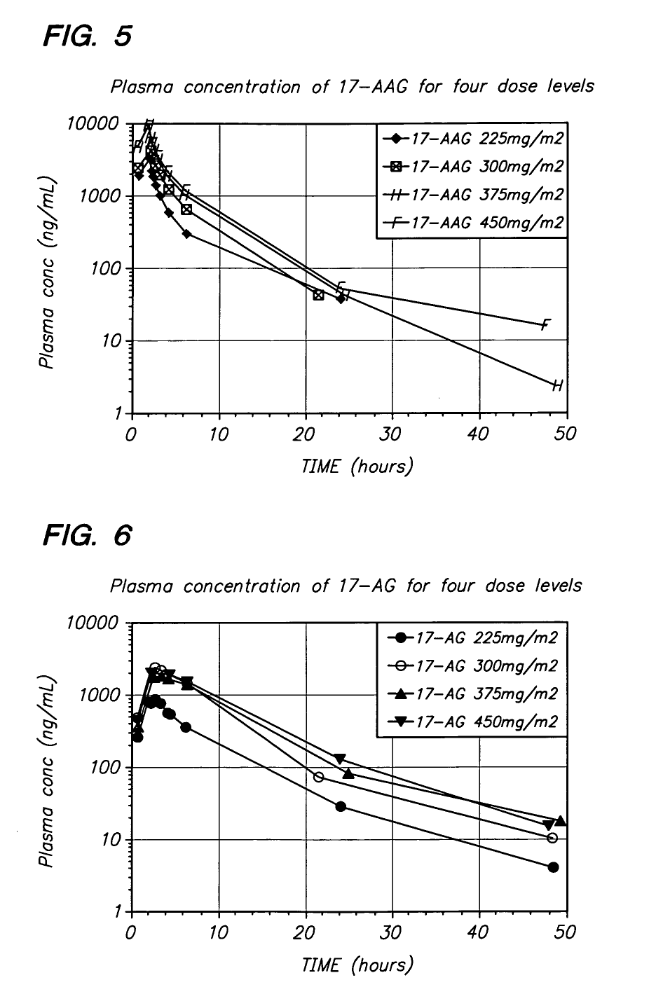

In general, the plasma concentrations of the metabolite (17-AG) were higher than for the parent compound (17-AAG) from approximately one hour post infusion. As the metabolite is biologically active, the actual exposure to drug is the sum of the two plasma profiles. FIGS. 2 to 4 show a more expected form of the curve. FIGS. 5 and 6 show the increase in concentration of 17-AAG and 17-AG, respectively, with increasing dose (mean).

For this dose range (225 to 450 mg/m²), the 17-AAG Cₘₐₓ increase was 3,258; 4,050; 9,405; and 8,107 ng/mL, and the 17-AG Cₘₐₓ increase was 957.8; 1,861; 2,250; and 2,225 ng/mL, for the 225, 300, 375, and 450 mg/m² dose levels, respectively. FIGS. 5 and 6 show the average increase in plasma concentration for 17-AAG and 17-AG, respectively, in relation to the dosages administered. There is a trend towards an asymptote of the plasma levels. Plasma concentration: time results were analyzed using non-compartmental methods to determine the PK of 17-AAG and 17-AG (Kinetica version 4.3; Innaphase, Champs sur Marne, France). Results are summarized in Tables 3 and 4.

TABLE 3
PK Parameters for 17-AAG
(Part I)

| Patient | Dose | Infusion | Cₘₐₓ | Tₘₐₓ | AUCₗₐₛₜ |
|---|---|---|---|---|---|
| Cohort | (mg) | duration (h) | (ng/mL) | (h) | (ng/mL*h) |
| Cohort 1 | | | | | |
| Mean | 403.8 | 2.0 | 3,257.5 | 1.8 | 7,951.6 |
| SD | 34.0 | 0.0 | 873.4 | 0.1 | 1,3787.5 |
| CV % | 8.4 | 2.4 | 26.8 | 7.9 | 17.3 |
| Cohort 2 | | | | | |
| Mean | 473.0 | 2.0 | 4,050.0 | 1.9 | 15,881.6 |
| SD | 26.2 | 0.0 | 718.8 | 0.1 | 2,015.1 |
| CV % | 5.5 | 0.0 | 17.7 | 5.9 | 12.7 |
| Cohort 3 | | | | | |
| Mean | 649 | 2.0 | 9,405 | 1.9 | 27,493 |
| SD | 76.0 | 0.1 | 29,37 | 0.1 | 9,481 |
| CV % | 11.7 | 4.8 | 31.2 | 6.9 | 34.5 |
| Cohort 4 | | | | | |
| Mean | 802 | 2 | 8,107 | 2 | 27,867 |
| SD | 86.2 | 0.1 | 1,987.6 | 0.6 | 10,422.4 |
| CV % | 10.8 | 5.1 | 24.5 | 35.1 | 37.4 |

(Part II)

| Patient | AUCₑₓₜᵣₐ | AUCₜₒₜₐₗ | AUCₑₓₜ | L_{z} | AUMCₜₒₜₐₗ |
|---|---|---|---|---|---|
| Cohort | (ng/mL*h) | (ng/mL*h) | (%) | (1/h) | ng/mL*(h²) |
| Cohort 1 | | | | | |
| Mean | 587.7 | 8,539.3 | 7.1 | 0.3804 | 29,168 |
| SD | 234.8 | 1,232.8 | 3.4 | 0.2 | 15,914 |
| CV % | 40.0 | 14.4 | 47.7 | 47.7 | 55 |
| Cohort 2 | | | | | |
| Mean | 280.9 | 16,163 | 1.6 | 0.1637 | 107,072 |
| SD | 196.3 | 2,194.1 | 1.1 | 0.1 | 68,147 |
| CV % | 69.9 | 13.6 | 66.5 | 47.5 | 64 |
| Cohort 3 | | | | | |
| Mean | 489.6 | 27,983 | 2.2 | 0.1996 | 124,179 |
| SD | 738.1 | 9,184 | 3.9 | 0.1 | 69,549 |
| CV % | 150.7 | 32.8 | 174.1 | 40.5 | 56 |
| Cohort 4 | | | | | |
| Mean | 558 | 28,425 | 2.8 | 0.2037 | 65,954 |
| SD | 736.9 | 10,043.5 | 4.5 | 0.11 | 71,407.3 |
| CV % | 132.1 | 35.3 | 162.7 | 53.7 | 108.3 |

(Part III)

| All Cohorts | BSA | t_{1/2} | MRT | Clearance | Clearance |
|---|---|---|---|---|---|
| Combined | (m²) | (h) | (h) | (L/h) | (L/h/m²) |
| Mean | 1.75 | 4.13 | 4.71 | 32.16 | 18.21 |
| SD | 0.18 | 2.48 | 2.09 | 12.16 | 6.09 |
| CV % | 10.51 | 60.16 | 44.35 | 37.82 | 33.46 |
| Min | 1.46 | 1.33 | 2.44 | 13.79 | 8.38 |
| Max | 2.12 | 11.42 | 11.44 | 58.82 | 29.71 |
| Median | 1.75 | 3.42 | 4.26 | 29.84 | 16.77 |

(Part IV)

| All Cohorts | V_{z} | V_{z} | Vₛₛ | Vₛₛ |
|---|---|---|---|---|
| Combined | (L) | (L/m²) | (L) | (L/m²) |
| Mean | 186.92 | 107.20 | 147.36 | 84.51 |
| SD | 156.38 | 89.31 | 94.80 | 54.00 |
| CV % | 83.66 | 83.32 | 64.33 | 63.89 |
| Min | 67.96 | 46.34 | 66.14 | 38.73 |
| Max | 791.84 | 444.85 | 549.56 | 308.74 |
| Median | 145.75 | 74.65 | 127.97 | 68.47 |

TABLE 4
PK Parameters for 17-AG

| | t_{1/2} | Cₘₐₓ | Tₘₐₓ | AUCₗₐₛₜ |
|---|---|---|---|---|
| Patient Cohort | (h) | (ng/mL) | (h) | (ng/ml*h) |
| Cohort 1 | | | | |
| Mean | 5.53 | 957.75 | 2.23 | 5,828 |
| SD | 1.08 | 305.95 | 0.17 | 2,362.22 |
| CV % | 19.58 | 31.94 | 7.45 | 40.53 |
| Cohort 2 | | | | |
| Mean | 5.97 | 1,861 | 2.47 | 13,261 |
| SD | 0.69 | 1,006 | 0.05 | 7,653.72 |
| CV % | 11.60 | 54.06 | 1.94 | 57.71 |
| Cohort 3 | | | | |
| Mean | 6.33 | 2,250 | 2.54 | 20,731 |
| SD | 0.84 | 521.3 | 0.36 | 7,887 |
| CV % | 13.26 | 23.17 | 14.23 | 38.05 |
| Cohort 4 | | | | |
| Mean | 5.89 | 2,224.6 | 2.84 | 19,817 |
| SD | 1.39 | 747.7 | 1.17 | 10,137 |
| CV % | 23.54 | 33.61 | 41.23 | 51.15 |

The terminal elimination half-lives for 17-AAG and 17-AG were 4.13±2.48 hours and 5.9±1.4 hours, respectively. The inclusion of the 48-hour post infusion blood sample did not change significantly the half-lives of either 17-AAG or 17-AG.

Total systemic clearance for 17-AAG was 32.16±12.16 L/h (or 18.21±6.09 L/h/m²). The distributive volumes for 17-AAG were: V_{z}=186.92±156.4 L (or 107.2±89.31 L/m²) and Vₛₛ=147.36±94.8 L (or 84.5±54.0 L/m²). The plasma results indicate th co-administration with trastuzumab had no effect on the kinetics of 17-AAG.

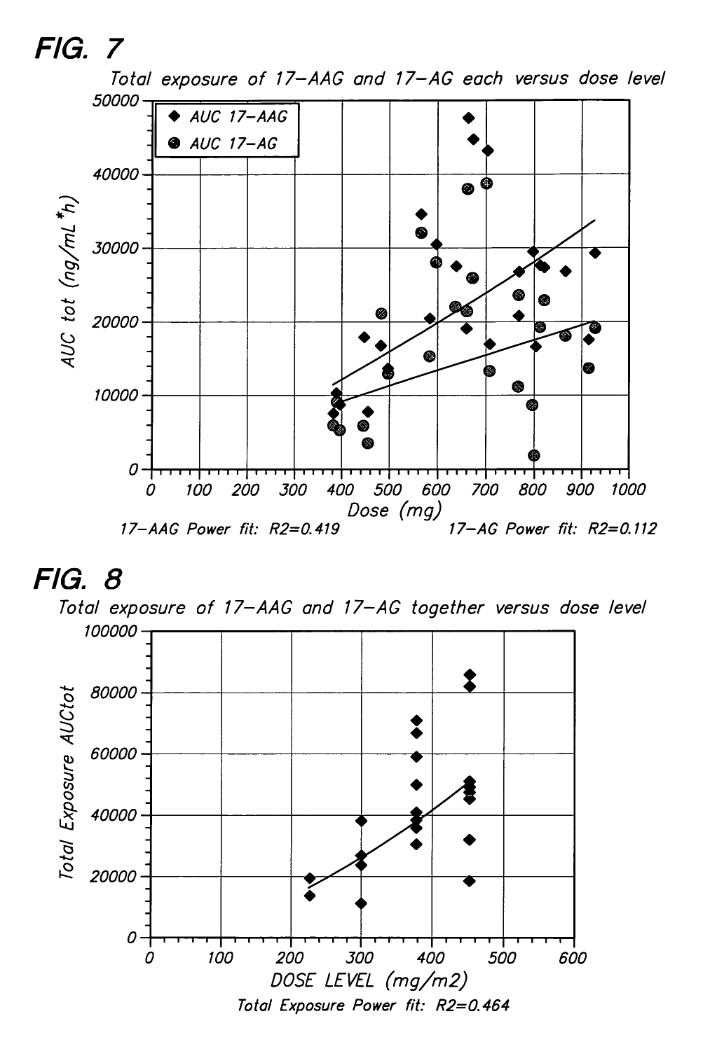

The total exposure of 17-AAG and 17-AG was determined by adding the AUCₜₒₜₐₗ values for 17-AAG and 17-AG. The mean ratio of 17-AG to 17-AAG was 73.3, with a standard deviation of 25.6, and the mean total exposure was 41,153, with a standard deviation of 20,513. FIG. 7 shows the plot of total exposure for both metabolite (17-AG) and parent drug (17-AAG) versus dose level. It shows a trend of increasing total exposure with increasing dose level. The lines have coefficients of determination (R²) equal to 0.419 and 0.112 for 17-AG and 17-AG, respectively. FIG. 8 shows the plot of total exposure for both metabolite (17-AG) and parent drug (17-AAG) added together versus dose level. It shows a trend of increasing total exposure with increasing dose level. The line has a coefficient of determination (R²) equal to 0.464.

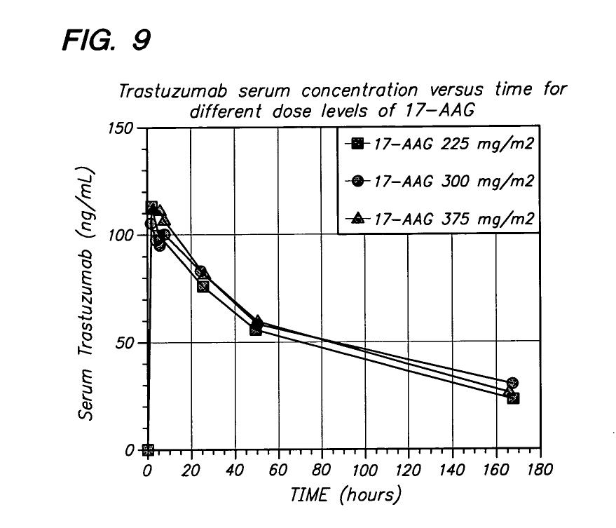

Measurement of the serum concentration of trastuzumab for patients at cohorts 1 to 3 indicated that the dose level of 17-AAG did not affect the PK of trastuzumab (FIG. 9). The overall PK of trastuzumab was determined to be as follows: Tₘₐₓ=3.5±2.4 hours, Cₘₐₓ=117 μg/mL, Clearance=22.91±7.69 mL/h, Vₛₛ=3.0±0.8 L, and AUCₜₒₜₐₗ=12,947±3,442 μg/m*h.

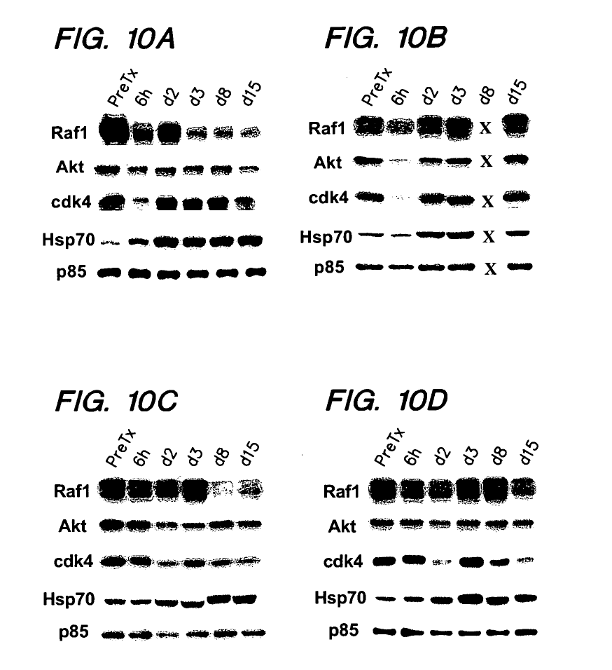

PD analysis. Peripheral blood leukocytes (PBL) were obtained from 6 patients prior to, and 6 hours, Day 2, Day 3, Day 8 (prior to Day 8 administration), and Day 15 (prior to Day 15 administration) after administration of 17-AAG and trastuzumab. The PBL were run on a protein gel and immunobloted for raf-1, AKT, cdk4, Hsp70 and p85. FIGS. 10A-10D show the results for four patients from cohort 3. Three of the patients were HER2+; the HER2 status of the fourth patient (FIG. 10B) was unknown. The results indicate that the treatment resulted in an induction of the heat stress response (as indicated by an increase in Hsp70 level) and increases in raf-1 levels for up to 3 days after infusion administration. The effect could still be observed one week post-dose.

Although the present invention has been described in detail with reference to specific embodiments, those of skill in the art will recognize that modifications and improvements are within the scope and spirit of the invention. Citation of publications and patent documents is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description are for purposes of illustration and not limitation of the following claims.

### REFERENCES

The following is a list of the references cited hereinabove. Each is incorporated by reference as if it had been specifically and individually incorporated by reference.

Adams et al. (2005a), US 2005/0227955 A1.

Adams et al. (2005b), US 2005/0288269 A1.

Bagatell et al. (2001), Clin. Cancer Res. 7 (7), 2076-2084, “Destabilization of Steroid Receptors by Heat Shock Protein 90-binding Drugs: A Ligand-independent Approach to Hormonal Treatment of Breast Cancer.”

Bagchi et al. (1997) U.S. Pat. No. 5,662,883.

Banerji et al. (2005) J. Clin. Oncol. 23(1):4152-4161, “Phase I Pharmacokinetic and Pharmacodynamic Study of 17-Allylamino, 17-Demethoxygeldanamycin in Patients with Advanced Malignancies.”

Boni et al. (1997) U.S. Pat. No. 5,683,715.

Bosch et al. (1996) U.S. Pat. No. 5,510,118.

Burger et al. (2004), Anticancer Drugs 15 (4), 377-87, “17-(Allylamino)17-demethoxygeldanamycin Activity in Human Melanoma.”

Carter et al. (2004), U.S. Pat. No. 6,713,485 B2.

Chen et al. (2005), Cancer Chemother. Pharmacol. 55:237-243, “Population pharmacokinetic analysis of 17-(allylamino)-17-demethoxygeldanamycin (17AAG) in adult patients with advanced malignancies.”

Cui et al. (2004), U.S. Pat. No. 6,677,368 B2.

De Bono and Rowinsky (2002), Trends Mol. Med. 8 (4) (Supp.), S19-26, “The ErbB receptor family: a therapeutic target for cancer.”

De Castro (1996) U.S. Pat. No. 5,534,270.

Desai et al. (2006) WO 2006/034147 A2.

Egorin et al. (1998), Cancer Res. 58:2385-2396, “Metabolism of 17-(allylamino)-17-demethoxygeldanamycin (NSC 330507) by murine and human hepatic preparations.”

Goetz et al. (2005), J. Clin. Oncol. 23(6):1078-1087, “Phase I trial of 17-allylamino-17-demethoxygeldanamycin in patients with advanced cancer.”

Genentech, Inc. (2005), Product brochure entitled “Your Herceptin® Therapy—How It Works and What to Expect.”

Greene et al. (2003), US 2003/0148932 A1.

Grem et al. (2005), J. Clin. Oncol. 23(9):1885-93, “Phase I and pharmacologic study of 17-(allylamino)-17-demethoxygeldanamycin in adult patients with solid tumors.”

Hilberg et al. (2003), US 2003/0162796 A1.

Hostein et al. (2001), Cancer Res. 61:4003-4009, “Inhibition of Signal Transduction by the Hsp90 Inhibitor 17-Allylamino-17-demethoxygeldanamycin Results in Cytostasis and Apoptosis.”

Hudziak et al. (1997), U.S. Pat. No. 5,677,171.

Hudziak et al. (1998a), U.S. Pat. No. 5,770,195.

Hudziak et al. (1998b), U.S. Pat. No. 5,772,997.

Hudziak et al. (2000), U.S. Pat. No. 6,165,464.

Hudziak et al. (2002a), U.S. Pat. No. 6,387,371 B1.

Hudziak et al. (2002b), U.S. Pat. No. 6,399,063 B1.

Isaacs et al. (2006) WO 2006/094029 A2.

Jiang and Shapiro. (2002), Proc. 93rd Ann Meet Am Assoc Cancer Res. Abstract 1645, “17-AAG induces Rb-dependent G1 arrest in lung cancer cell lines.”

Johnson, Jr., et al., U.S. patent application Ser. No. 11/412,298, filed Apr. 26, 2006.

Johnson, Jr., et al. U.S. patent application Ser. No. 11/412,299, filed Apr. 26, 2006.

Johnston (2006), Drugs Today 42 (7), 441, “Lapatinib: A novel EGFR/HER2 tyrosine kinase inhibitor for cancer.”

Lambert et al. (2000) WO 00/71163.

Mansfield et al. (2006) US 2006/0067953 A1.

Munster et al. (2001), Proc. Am. Soc. Clin. Oncol. 20, Abstract 327, “Phase I Trial of 17-(allylamino)-17-Demethoxygeldanamycin (17-AAG) in Patients (Pts) with Advanced Solid Malignancies.”

National Cancer Institute (2003) Common Terminology Criteria for Adverse Events v3.0, Dec. 12, 2003 (CTCAE).

Nguyen et al. (2000), Ann. Thorac. Surg. 70:1853-1860, “Modulation of metastasis phenotypes of non-small cell lung cancer cells by 17-allylamino 17-demethoxy geldanamycin.”

Nimmanapalli et al. (2001), Cancer Res. 61:1799-1804, “Geldanamycin and Its Analogue 17-Allylamino-17-demethoxygeldanamycin Lowers Bcr-Abl Levels and Induces Apoptosis and Differentiation of Bcr-Abl-positive Human Leukemic Blasts.”

Park et al. (2000), Nature Biotechn. 18, 1194-198, “Rationally designed anti-HER2/neu peptide mimetic disables P185^{HER2/neu} tyrosine kinase in vitro and in vivo.”

Quay et al. (1998), WO 98/30205.

Rahman et al. (1995) U.S. Pat. No. 5,424,073.

Ridolfi et al. (2000), Mod. Pathol. 13 (8), 866-873, “HER-2/neu testing in breast carcinoma: a combined immunohistochemical and fluorescence in situ hybridization approach.”

Sasaki et al. (1979), J. Antibiotics 32 (8), 849-851, “Growth inhibition of virus transformed cells in vitro and antitumor activity in vivo of geldanamycin and its derivatives.”

Sasaki et al. (1981), U.S. Pat. No. 4,261,989.

Schnur (1995), U.S. Pat. No. 5,387,584.

Schnur et al. (1995a), J. Med. Chem., 38, 3806-3812, “Inhibition of Oncogene Products p185 (erbB-2) in Vitro and in Vivo by Geldanamycin and Dihydrogeldanamycin Derivatives”

Schnur et al. (1995b), J. Med. Chem., 38, 3813-3820, “erbB-2 Oncogene Inhibition by Geldanamycin Derivatives: Synthesis, Mechanism of Action, and Structure-Activity Relationships”

Schnur et al. (1999), U.S. Pat. No. 5,932,566.

Schulte and Neckers. (1998), Cancer Chemother. Pharmacol. 42, 273-279, “The benzoquinone ansamycin 17-allylamino-17-demethoxygeldanamycin binds to HSP90 and shares important biologic activities with geldanamycin.”

Sonntag et al. (2001) WO 01/10412 A1.

Straubinger et al. (1995) U.S. Pat. No. 5,415,869.

Tabibi et al. (2004) U.S. Pat. No. 6,682,758 B1.

Tang et al. (2000a), U.S. Pat. No. 6,103,728.

Tang et al. (2000b), U.S. Pat. No. 6,133,305.

Therasse et al. (2000), J. Nat. Cancer Inst. 92 (3), 205-216, “New Guidelines to Evaluate the Response to Treatment in Solid Tumors.”

Ulm et al. (2003) WO 03/086381 A1.

Ulm et al. (2004) WO 2004/082676 A1.

Vite et al. (2005), U.S. Pat. No. 6,916,815.

Wermuth (2003), “Designing Prodrugs and Bioprecursors,” in Wermuth, ed., The Practice of Medicinal Chemistry, 2nd Ed., pp. 561-586 (Academic Press 2003).

Wilson et al. (2001), Proc. Am. Soc. Clin. Oncol. 20, abstract 325, “Phase I Pharmacolologic Study of 17-(Allylamino)-17-Demethoxygeldanamycin (AAG) in Adult Patients with Advanced Solid Tumors.”

Xia et al. (2005), Oncogene 24 (41), 6213-6221, “Combining lapatinib (GW 572016), a small molecule inhibitor of ErbB1 and ErbB2 tyrosine kinases, with therapeutic anti-ErbB2 antibodies enhances apoptosis of ErbB2-overexpressing breast cancer cells.”

Zhong et al. (2005) US 2005/0256097 A1.

What is claimed is:

1. A method for treating breast cancer in a subject in need of such treatment, said method comprising the step of administering to said subject a therapeutically effective dose of 17-allylamino-17-demethoxy-geldanamycin (17-AAG) or 17-amino-17-demethoxy-geldanamycin (17-AG) or a prodrug of either 17-AAG or 17-AG, and a therapeutically effective dose of a HER2 inhibitor, and optionally repeating said step until no further therapeutic benefit is obtained.

2. A method for treating breast cancer in a subject in need of such treatment, said method comprising the step of administering multiple doses of 17-AAG or a prodrug thereof to said patient over a time period of at least once a week, wherein each such dose is in the range of about 300 mg/m² to about 450 mg/m² of 17-AAG, or an equivalent amount of a 17-AAG prodrug or 17-AG prodrug, and multiple doses of said HER2 inhibitor, wherein said HER2 inhibitor is trastuzumab and each such dose is in range of about 2 mg/kg to about 4 mg/kg.

3. The method of claim 2, wherein said dose is administered once weekly for at least four weeks.

4. The method of claim 2, wherein each such dose is in the range of about 375 mg/m² to about 450 mg/m² of 17-AAG, or an equivalent amount of a 17-AAG prodrug or 17-AG prodrug.

5. The method of claim 4, wherein said dose is administered once weekly for at least four weeks.

6. The method of claim 2, wherein each such dose is in the range of about 450 mg/m² of 17-AAG, or an equivalent amount of a 17-AAG prodrug or 17-AG prodrug.

7. The method of claim 6, wherein said dose is administered once weekly for at least four weeks.

8. The method of claim 1, wherein the prodrug of either 17-AAG or 17-AG is 17-allylamino-18,21-dihydro-17-demethoxygeldanamycin.

9. The method of claim 1, wherein the HER2 inhibitor is a monoclonal antibody.

10. The method of claim 1, wherein the HER2 inhibitor is trastuzumab.

11. The method of claim 1, wherein the HER2 inhibitor is a dual tyrosine kinase inhibitor.

12. The method of claim 1, wherein the breast cancer is HER2-positive breast cancer.

13. The method of claim 1, further comprising the step of testing the subject for HER2 overexpression prior to the adminstering step.

14. A method for treating breast cancer in a subject in need of such treatment, said method comprising the step of administering to said subject a therapeutically effective dose of a HER2 inhibitor and a therapeutically effective dose of 17-AAG or a prodrug of 17-AAG that results in an AUCₜₒₜₐₗ of 17-AAG per dose in the range of about 13,000 ng/mL*h to about 48,000 ng/mL*h.

15. The method of claim 14, wherein said dose of 17-AAG or a prodrug of 17-AAG is administered at a rate and frequency such that the C_{max of} 17-AAG does not exceed 14,000 ng/mL.

16. The method of claim 14, wherein said dose of 17-AAG or a prodrug of 17-AAG is administered at a rate and frequency such that the Cₘₐₓ of 17-AAG is greater than 3,600 ng/mL.

17. The method of claim 16, wherein said Cₘₐₓ of 17-AAG is greater than 5,000 ng/mL.

18. The method of claim 16, wherein said dose of 17-AAG or a prodrug of 17-AAG is administered at a rate and frequency such that the Cₘₐₓ of 17-AAG is greater than 3,600 ng/mL but does not exceed 14,000 ng/mL.

19. The method of claim 18, wherein said Cₘₐₓ of 17-AAG is greater than 5,000 ng/mL but does not exceed 14,000 ng/mL.

20. A method of treating breast cancer in a subject in need of such treatment, said method comprising the step of administering to said subject a therapeutically effective dose of a HER2 inhibitor and a therapeutically effective dose of 17-AG or a prodrug of 17-AG that results in an AUCₜₒₜₐₗ of 17-AG per dose in the range of about 5,800 ng/mL*h to about 39,000 ng/mL*h.

21. The method of claim 20, wherein said dose of 17-AG or a prodrug of 17-AG is administered at a rate and frequency such that the Cₘₐₓ of 17-AG does not exceed 3,300 ng/mL.

22. The method of claim 20, wherein said dose of 17-AG or a prodrug of 17-AG is administered at a rate and frequency such that the Cₘₐₓ of 17-AG is greater than 800 ng/mL.

23. The method of claim 22, wherein said dose of 17-AG or a prodrug of 17-AG is administered at a rate and frequency such that the Cₘₐₓ of 17-AG is greater than 1,100 ng/mL.

24. The method of claim 22, wherein said dose of 17-AG or a prodrug of 17-AG is administered at a rate and frequency such that the Cₘₐₓ of 17-AG is greater than 800 ng/mL but does not exceed 3,300 ng/mL.

25. The method of claim 24, wherein said Cₘₐₓ of 17-AG is greater than 1,100 ng/mL but does not exceed 3,300 ng/mL.

26. A method for treating breast cancer in a subject in need of such treatment, said method comprising the step of administering to said subject a therapeutically effective dose of a HER2 inhibitor and a therapeutically effective dose of 17-AAG, a prodrug of 17-AAG, 17-AG, or a prodrug of 17-AG that results in a combined AUCₜₒₜₐₗ of 17-AAG and 17-AG per dose in the range of about 23,000 ng/mL*h to about 82,000 ng/mL*h.

27. The method of claim 26, wherein said dose of 17-AAG, a prodrug of 17-AAG, 17-AG, or a prodrug of 17-AG is administered at a rate and frequency such that the Cₘₐₓ of 17-AAG does not exceed 14,000 ng/mL or said Cₘₐₓ of 17-AG does not exceed 3,300 ng/mL.

28. The method of claim 26, wherein said dose of 17-AAG, a prodrug of 17-AAG, 17-AG, or a prodrug of 17-AG, is administered at a rate and frequency such that the Cₘₐₓ of 17-AAG is greater than 3,600 ng/mL or the Cₘₐₓ of 17-AG is greater than 800 ng/mL.

29. The method of claim 28, wherein said Cₘₐₓ of 17-AAG is greater than 5,000 ng/mL or the Cₘₐₓ of 17-AG is greater than 1,100 ng/mL.

30. The method of claim 26, wherein said dose of 17-AAG, a prodrug of 17-AAG, 17-AG, or a prodrug of 17-AG, is administered at a rate and frequency such that the Cₘₐₓ of 17-AAG is greater than 3,600 ng/mL but does not exceed 14,000 ng/mL or the Cₘₐₓ of 17-AG is greater than 800 ng/mL but does not exceed 3,300 ng/mL.

31. The method of claim 30, wherein said Cₘₐₓ of 17-AAG is greater than 5,000 ng/mL or said Cₘₐₓ of 17-AG is greater than 1,100 ng/mL.

32. A method of treating breast cancer in a subject in need of such treatment, said method comprising the step of administering to said subject a therapeutically effective dose of a HER2 inhibitor and a therapeutically effective dose of 17-AAG or a prodrug of 17-AAG that results in a Terminal T_{1/2} of 17-AAG in the range of 1.5 h to 12 h.

33. The method of claim 32, wherein said dose of 17-AAG or a prodrug of 17-AAG results in an AUCₜₒₜₐₗ of 17-AAG per dose in the range of about 13,000 ng/mL*h to about 48,000 ng/mL*h.

34. A method for treating breast cancer in a subject in need of such treatment, said method comprising the step of administering to said subject a therapeutically effective dose of a HER2 inhibitor and a therapeutically effective dose of 17-AG or a prodrug of 17-AG that results in a Terminal T_{1/2} of 17-AG in the range of 3.7 h to 8.8 h.

35. The method of claim 34, wherein said dose of 17-AG or a prodrug of 17-AG administered results in an AUCₜₒₜₐₗ of 17-AG per dose in the range of about 5,800 ng/mL*h to about 39,000 ng/mL*h.

36. A method for treating breast cancer in a subject in need of such treatment, said method comprising the step of administering to said subject a therapeutically effective dose of a HER2 inhibitor and a therapeutically effective dose of 17-AAG or a prodrug of 17-AAG that results in a Volume of distribution V_{Z} of 17-AAG in the range of 67 L to 800 L.

37. The method of claim 36, wherein said dose of 17-AAG or a prodrug of 17-AAG administered results in an AUCₜₒₜₐₗ of 17-AAG per dose in the range of about 13,000 ng/mL*h to about 48,000 ng/mL*h.

38. A method for treating breast cancer in a subject in need of such treatment, said method comprising the step of administering to said subject a therapeutically effective dose of a HER2 inhibitor and a therapeutically effective dose of 17-AAG or a prodrug of 17-AAG that results in a Clearance of 17-AAG in the range of 13 L/h to 52 L/h.

39. The method of claim 38, wherein said dose of 17-AAG or a prodrug of 17-AAG administered results in an AUCₜₒₜₐₗ of 17-AAG per dose in the range of about 13,000 ng/mL*h to about 48,000 ng/mL*h.

40. A method for treating breast cancer in a subject in need of such treatment, said method comprising the step of administering to said subject a therapeutically effective dose of a HER2 inhibitor and a therapeutically effective dose of 17-AAG or a prodrug of 17-AAG that results in a Volume of distribution Vₛₛ of 17-AAG in the range of 66 L to 550 L.

41. The method of claim 40, wherein said dose of 17-AAG or a prodrug of 17-AAG administered results in an AUCₜₒₜₐₗ of 17-AAG per dose in the range of about 13,000 ng/mL*h to about 48,000 ng/mL*h.
